(19) United States
(12) Patent Application Publication
(10) Pub. No.: US 2010/0041997 A1
(43) Pub. Date: Feb. 18, 2010

(54) ULTRASONIC IMAGING APPARATUS

(76) Inventors: Shinichi Amemiya, Tokyo (JP); Per Arne Linnerud, Horten (NO); Roar Waaler, Tonsberg (NO)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/536,299

(22) Filed: Aug. 5, 2009

(30) Foreign Application Priority Data
Aug. 13, 2008 (JP) 2008-208503

(51) Int. Cl. A61B 8/14

(52) U.S. Cl. 600/459

(57) ABSTRACT

An ultrasonic imaging apparatus includes a pulsar including an output line coupled to a piezoelectric transducer and a plurality of first push-pull circuits whose output units are coupled to the output line, and a power source unit which supplies a plurality of power source voltages with different levels to the first push-pull circuits. At least one of the first push-pull circuits includes first rectification elements which prevent reverse current from flowing into first complementary transistors configuring the first push-pull circuit. The pulsar includes a second push-pull circuit whose output unit is coupled to the output line, the same power source voltage as in the first push-pull circuit with the first rectification elements is applied to the second push-pull circuit, and current in the direction opposite to that in the first push-pull circuit flows in the second push-pull circuit by turning on second complementary transistors configuring the second push-pull circuit.

### CROSS REFERENCE TO RELATED APPLICATIONS

This application claims the benefit of Japanese Patent Application No. 2008-208503 filed Aug. 13, 2008, which is hereby incorporated by reference in its entirety.

### BACKGROUND OF THE INVENTION

### Technical Field

The embodiments described herein relate to an ultrasonic imaging apparatus including a pulsar that generates an electric signal for driving a piezoelectric transducer.

Recently, a burst waveform including a plurality of same waveforms is used in an electric signal that drives a piezoelectric transducer for generating an ultrasonic wave in an ultrasonic imaging apparatus (refer to Japanese Patent Application Laid-Open No. 2000-005169). The burst waveform has a frequency of about 3 to 10 MHz corresponding to the resonance frequency of the piezoelectric transducer, and has an amplitude voltage of about 100V. The number of piezoelectric transducers which are driven at the same time reaches tens of channels, and the ultrasonic imaging apparatus features compactness, so that it is preferable that a transmission unit for generating these burst waveforms has a simple configuration.

As the transmission unit with a simple configuration for generating the burst waveform, there exist multilevel pulsars in which push-pull circuits having different levels of power source voltage are coupled in parallel. The multilevel pulsars switch output voltage stepwise by turning on or off the push-pull circuits to simply generate the burst waveforms composed of pseudo sine waves similar to sine waves.

However, according to the above-described background art, when the output voltage is switched stepwise, power loss occurs. Specifically, when the output voltage of the multilevel pulsar is switched stepwise, there occurs charging and discharging of electric charge charged to the piezoelectric transducer with capacitive electric characteristics. The charging and discharging occurs between the piezoelectric transducer and a ground resistor coupled in parallel to the capacitive piezoelectric transducer, and causes the occurrence of the power loss.

In particular, the power loss causes heat generation, and becomes a significant level for the ultrasonic imaging apparatus for driving multi-channels.

It is desirable that the problem described previously is solved.

### BRIEF DESCRIPTION OF THE INVENTION

An ultrasonic imaging apparatus, according to a first aspect of the invention, which supplies predetermined voltage to a piezoelectric transducer to transmit an ultrasonic wave, the apparatus including: a pulsar including an output line coupled to the piezoelectric transducer and a plurality of first push-pull circuits whose output units are coupled to the output line; and a power source unit which supplies a plurality of power source voltages with different levels to the plurality of first push-pull circuits, wherein at least any one of the plurality of first push-pull circuits includes first rectification elements which prevent reverse current from flowing into first complementary transistors configuring the first push-pull circuit, and the pulsar includes a second push-pull circuit whose output unit is coupled to the output line, the same power source voltage as in the first push-pull circuit with the first rectification elements is applied to the second push-pull circuit, and current in the direction opposite to that in the first push-pull circuit flows in the second push-pull circuit by turning on second complementary transistors configuring the second push-pull circuit.

In the invention according to the first aspect, the current in the direction opposite to that in the first push-pull circuit flows in the second push-pull circuit by turning on the second complementary transistors configuring the second push-pull circuit.

The ultrasonic imaging apparatus according to a second aspect of the invention, wherein the power source unit supplies the maximum driving voltage among the plurality of power source voltages to the first push-pull circuit without the first rectification elements.

In the invention according to the second aspect, the first push-pull circuit with the maximum driving voltage is not provided with the first rectification elements.

The ultrasonic imaging apparatus according to a third aspect of the invention, wherein the first push-pull circuits include, as the first complementary transistors, P-channel first Field Effect Transistors on the high voltage side of the power source voltage relative to the output units, and N-channel Field Effect Transistors on the low voltage side of the power source voltage relative to the output units in the ultrasonic imaging apparatus described in the first or second aspect.

The ultrasonic imaging apparatus according to a fourth aspect of the invention, wherein the second push-pull circuits includes, as the second complementary transistors, an N-channel second Field Effect Transistor on the high voltage side relative to the output unit coupled to the output line, and a P-channel Field Effect Transistor on the low voltage side relative to the output unit, and further includes second rectification elements which are coupled in series to the respective second Field Effect Transistors in the ultrasonic imaging apparatus described in the third aspect.

In the invention according to the fourth aspect, the N-channel or P-channel polarity of the complementary transistor of the second push-pull circuit is opposite to that of the complementary transistor of the first push-pull circuit, and the second rectification elements of the output unit limits the direction in which the current flows.

The ultrasonic imaging apparatus according to a fifth aspect of the invention, wherein each of the second rectification elements which are coupled in series to the respective N-channel second Field Effect Transistors is a diode which is coupled in the forward direction from the output unit toward the power source unit, and each of the second rectification elements which are coupled in series to the respective P-channel second Field Effect Transistors is a diode which is coupled in the forward direction from the power source unit toward the output unit in the ultrasonic imaging apparatus described in the fourth aspect.

In the invention according to the fifth aspect, the current is allowed to flow in the second push-pull circuit in the direction different from that flowing in the complementary transistors of the first push-pull circuit with the first rectification elements.

The ultrasonic imaging apparatus according to a sixth aspect of the invention, including a pulsar control unit which turns on or off the first Field Effect Transistors and the second Field Effect Transistors in the ultrasonic imaging apparatus described in the fourth or fifth aspect.

In the invention according to the sixth aspect, the first and second push-pull circuits are controlled by the pulsar control unit.

The ultrasonic imaging apparatus according to a seventh aspect of the invention, wherein the pulsar control unit includes a first driver which turns on or off the first Field Effect Transistors and a second driver which turns on or off the second Field Effect Transistors in the ultrasonic imaging apparatus described in the sixth aspect.

In the invention according to the seventh aspect, the first Field Effect Transistors and the second Field Effect Transistors are turned on or off by the first and second drivers which are different from each other.

The ultrasonic imaging apparatus according to an eighth aspect of the invention, wherein the pulsar control unit includes a pseudo sine wave generation device which outputs the plurality of power source voltages to the output line in a sine wave shape by turning on or off the first Field Effect Transistors in a predetermined order in the ultrasonic imaging apparatus described in the sixth or seventh aspect.

In the invention according to the eighth aspect, waveforms similar to the sine waves are simply generated by the pseudo sine wave generation device.

The ultrasonic imaging apparatus according to a ninth aspect of the invention, wherein in synchronization with turning-off of the N-channel or P-channel first Field Effect Transistor of the first push-pull circuit without the first rectification elements and turning-on of the N-channel or P-channel first Field Effect Transistor of the first push-pull circuit with the first rectification elements, the pseudo sine wave generation device turns on the N-channel or P-channel second Field Effect Transistor of the second push-pull circuit in the ultrasonic imaging apparatus described in the eighth aspect.

In the invention according to the ninth aspect, in synchronization with turning-on of the first push-pull circuit with the first rectification elements, the corresponding second Field Effect Transistor of the second push-pull circuit with the second rectification elements is turned on.

The ultrasonic imaging apparatus according to a tenth aspect of the invention, wherein in synchronization with turning-off of the N-channel or P-channel first Field Effect Transistor of the first push-pull circuit with the first rectification elements, the pseudo sine wave generation device turns off the N-channel or P-channel second Field Effect Transistor of the second push-pull circuit in the ultrasonic imaging apparatus described in the ninth aspect.

In the invention according to the tenth aspect, when the N-channel or P-channel first Field Effect Transistor of the first push-pull circuit with the first rectification elements is turned on, the N-channel or P-channel second Field Effect Transistor of the second push-pull circuit is turned on.

The ultrasonic imaging apparatus according to an eleventh aspect of the invention, wherein after a predetermined period of time passes since the N-channel or P-channel first Field Effect Transistor of the first push-pull circuit without the first rectification elements is turned off and the N-channel or P-channel first Field Effect Transistor of the first push-pull circuit with the first rectification elements is turned on, the pseudo sine wave generation device turns off the N-channel or P-channel second Field Effect Transistor of the second push-pull circuit in the ultrasonic imaging apparatus described in the ninth aspect.

In the invention according to the eleventh aspect, the second Field Effect Transistor is turned off after a predetermined period of time passes since the N-channel or P-channel first Field Effect Transistor of the first push-pull circuit with the first rectification elements is turned on.

The ultrasonic imaging apparatus according to a twelfth aspect of the invention, wherein the pulsar control unit turns on or off only the N-channel or P-channel first Field Effect Transistor of the first push-pull circuit without the first rectification elements to output the power source voltages to the output line in a rectangular shape, and in this case, the pulsar control unit does not turn on or off the N-channel or P-channel second Field Effect Transistor of the second push-pull circuit in the ultrasonic imaging apparatus described in the sixth aspect.

In the invention according to the twelfth aspect, the second push-pull circuit is not operated.

The ultrasonic imaging apparatus according to a thirteenth aspect of the invention, wherein the second Field Effect Transistors are lower in the maximum rating of drain current flowing between drains and sources, as compared to the first Field Effect Transistors in the ultrasonic imaging apparatus described in any one of the fourth to twelfth aspects.

In the invention according to the thirteenth aspect, the shapes of the second Field Effect Transistors are made small, and increase in size caused by adding the transistors is suppressed.

The ultrasonic imaging apparatus according to a fourteenth aspect of the invention, wherein the power source unit generates the power source voltages with the same level and with the positive and negative voltage polarities in the ultrasonic imaging apparatus described in any one of the first to thirteenth aspects.

In the invention according to the fourteenth aspect, the electric signal for driving the piezoelectric transducer is made stable so as to oscillate relative to the ground potential as a center.

The ultrasonic imaging apparatus according to a fifteenth aspect of the invention, wherein the pulsar includes a ground circuit which turns on or off the connection between the output line and a ground terminal in the ultrasonic imaging apparatus described in any one of the first to fourteenth aspects.

In the invention according to the fifteenth aspect, the ground potential of the electric signal for driving the piezoelectric transducer is made secured.

According to the embodiments described herein, the consumption of the stable current generated by the pulsar that forms the pseudo sine waves is eliminated, the power consumption generated in a transition state in which the voltage is changed can be reduced, and the heat generation of the pulsar can be reduced.

Further objects and advantages of the present invention will be apparent from the following description of embodiments of the invention as illustrated in the accompanying drawings.

### BRIEF DESCRIPTION OF THE DRAWINGS

FIG. 1 is a block diagram for showing the entire configuration of an ultrasonic imaging apparatus.

FIG. 2 is a block diagram for showing a configuration of an image production unit of the ultrasonic imaging apparatus.

FIG. 3 is a block diagram for showing a configuration of a transmission unit of the ultrasonic imaging apparatus.

FIG. 4 is a circuit diagram for showing a configuration of a multilevel pulsar that may be used with the ultrasonic imaging apparatus.

FIGS. 5(A) and 5(B) are explanatory diagrams for showing the entire output operation of the multilevel pulsar.

FIG. 6 is an explanatory diagram for showing a circuit operation of the multilevel pulsar.

FIGS. 7(A) and 7(B) are explanatory diagrams for showing output voltage of the multilevel pulsar and current flowing into transistors.

FIG. 8 is an explanatory diagram for showing a configuration and an operation of a multilevel pulsar with a simple configuration.

FIGS. 9(A) and 9(B) are explanatory diagrams for showing an operation and changes of current when the output voltage of the multilevel pulsar with a simple configuration is switched.

### DETAILED DESCRIPTION OF THE INVENTION

Embodiments of an ultrasonic imaging apparatus according to the invention will be described below with reference to the accompanying diagrams. It should be noted that the invention is not limited to the embodiments described herein.

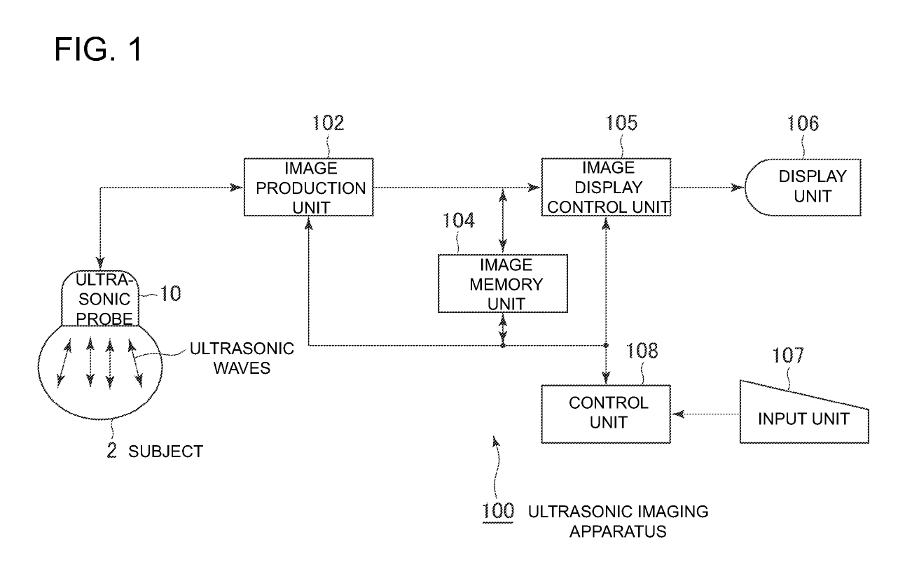

First of all, the entire configuration of an ultrasonic imaging apparatus 100 according to the embodiment will be described. FIG. 1 is a block diagram for showing the entire configuration of the ultrasonic imaging apparatus 100 according to the embodiment. The ultrasonic imaging apparatus 100 includes an ultrasonic probe 10, an image production unit 102, an image memory unit 104, an image display control unit 105, a display unit 106, an input unit 107, and a control unit 108.

The ultrasonic probe 10 is equipped with a piezoelectric transducer array, and transmits and receives ultrasonic waves. The ultrasonic probe 10 closely-attached to a surface of a subject 2 irradiates the ultrasonic waves onto an imaging cross section, and receives an ultrasonic echo reflected each time from the inside of the subject 2 as a time-series sound ray. The ultrasonic probe 10 performs electronic scanning while sequentially switching the irradiation direction of the ultrasonic waves.

The image production unit 102 generates an electric signal for driving the piezoelectric transducer array of the ultrasonic probe, and performs a B-mode process or a Doppler process using the electric signal received by the piezoelectric transducer array to form B-mode image information or Doppler image information. The detailed functions of the image production unit 102 will be described later.

The image memory unit 104 includes a large-capacity memory, and stores two-dimensional tomographic image information and cine image information that is two-dimensional tomographic image information that is temporally changed.

The image display control unit 105 performs display frame rate conversion of the B-mode image information generated by the B-mode process and blood flow image information generated by the Doppler process, and controls the shape and position of the image display.

The display unit 106 includes a CRT (Cathode Ray Tube), an LCD (Liquid Crystal Display) or the like, and displays the B-mode image or the blood flow image.

The input unit 107 includes a keyboard or the like, and operation information is input by an operator. For example, operation information for selecting display in the B-mode or display of the Doppler process, and operation information for setting a Doppler imaging area to perform the Doppler process are input through the input unit 107.

The control unit 108 controls the operation of the respective units of the ultrasonic imaging apparatus including the ultrasonic probe on the basis of the operation information input through the input unit 107 and a program and data that are stored in advance.

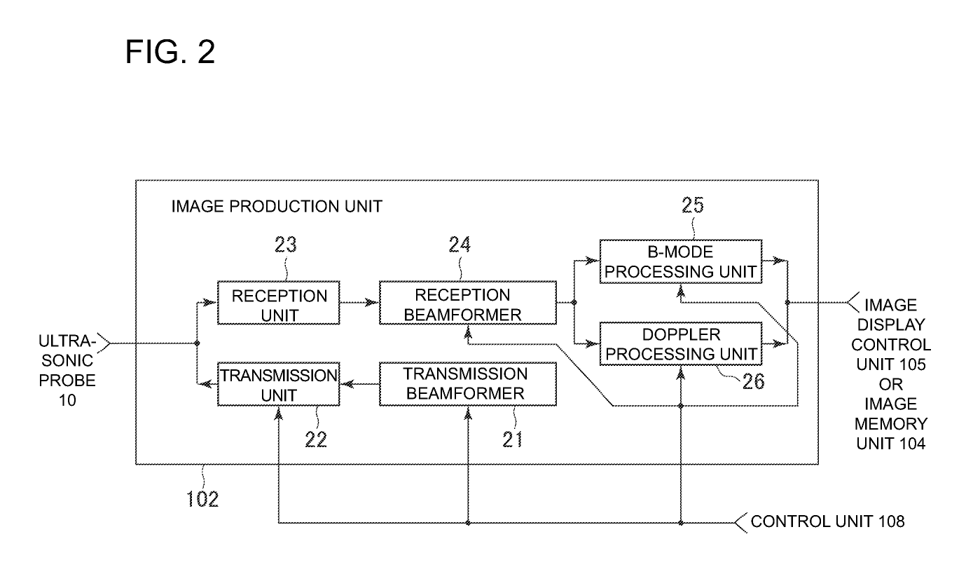

FIG. 2 is a block diagram for showing a configuration of the image production unit 102. The image production unit 102 includes a transmission beamformer 21, a transmission unit 22, a reception unit 23, a reception beamformer 24, a B-mode processing unit 25, and a Doppler processing unit 26. The transmission beamformer 21 generates a driving signal with a predetermined delay time so as to perform electronic focus at a set focal depth position on the basis of information from the control unit 108.

The transmission unit 22 forms a burst waveform that drives the piezoelectric transducer of the ultrasonic probe 10 on the basis of the driving signal from the transmission beamformer 21. It should be noted that the transmission unit 22 is described later in detail.

The reception unit 23 performs initial amplification of the electric signal received by the piezoelectric transducer of the ultrasonic probe 10. The reception beamformer 24 performs delay addition in which the predetermined delay time similar to that at the time of transmission is added to the electric signal received by the reception unit 23, and forms an electric signal on the sound ray.

The B-mode processing unit 25 performs processes such as logarithmic conversion and a filter process for the electric signal on the sound ray in which the delay time is added, and forms the B-mode image. The Doppler processing unit 26 performs orthogonal detection, a filter process and the like for the electric signal on the sound ray in which the delay time is added, and displays the blood flow information in the subject 2 as frequency spectrum information or CMF (Colour Flow Mapping) information.

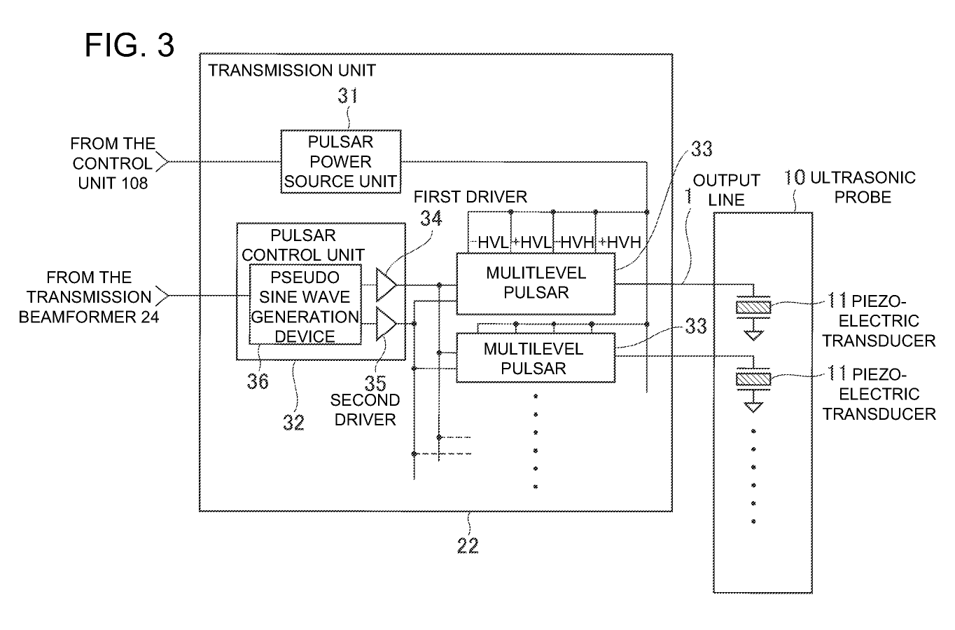

FIG. 3 is a block diagram for showing a configuration of the transmission unit 22. The transmission unit 22 includes a pulsar power source unit 31, a pulsar control unit 32, and a plurality of multilevel pulsars 33. The pulsar control unit 32 includes a first driver 34, a second driver 35, and a pseudo sine wave generation device 36, and allows the multilevel pulsars 33 to generate predetermined driving waveforms on the basis of the driving signal from the transmission beamformer 21. The driving waveform includes a rectangular wave or a pseudo sine wave, and in the case of generating, for example, the pseudo sine wave, a controlling signal is formed by the pseudo sine wave generation device 36.

The first driver 34 and the second driver 35 include a plurality of drivers (not shown), and drive transistors Q1 to Q8 to be described later. It should be noted that the second driver 35 is a driver that is lower in the maximum rating of output current and the driving capability than the first driver 34.

The pulsar power source unit 31 is a high-voltage power source unit that is configured by using a switching regulator or the like. The pulsar power source unit 31 generates positive and negative maximum driving voltages ±HVH corresponding to the maximum amplitude of the pseudo sine wave to be generated and positive and negative intermediate driving voltages ±HVL with the approximately half the levels of the maximum driving voltages ±HVH.

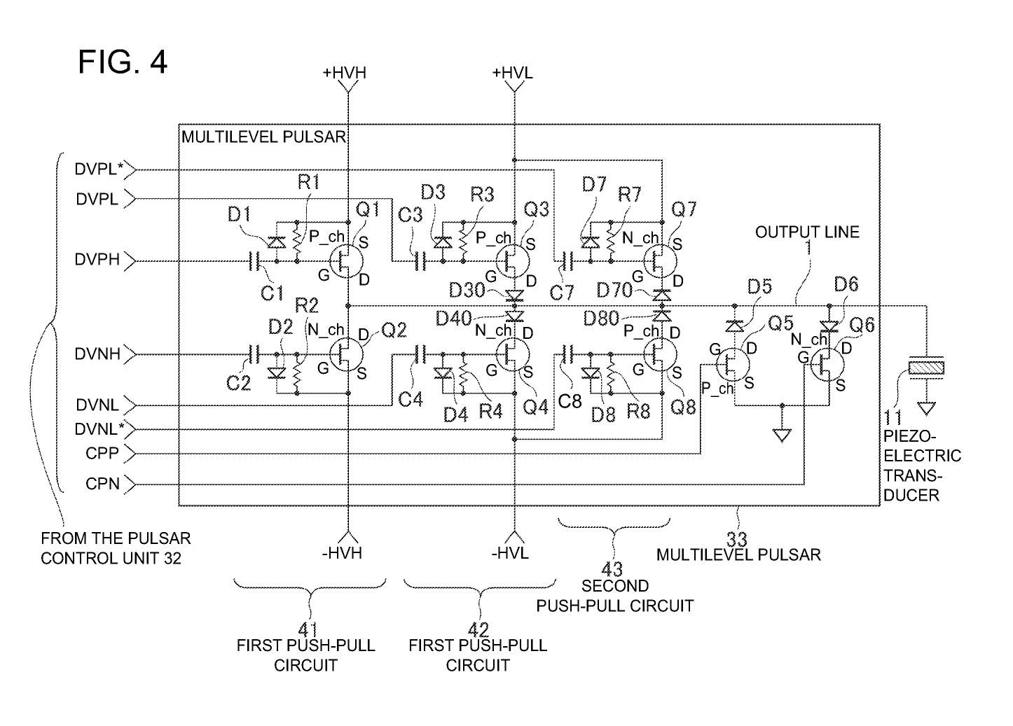

The multilevel pulsars 33 generate the rectangular waves or the pseudo sine waves on the basis of the controlling signal from the pulsar control unit 32. FIG. 4 is a circuit diagram for showing a configuration of the multilevel pulsar 33. The multilevel pulsar 33 includes an output line 1 made of an electric conductor coupled to the piezoelectric transducer 11, transistors Q1 to Q8, diodes D1 to D8, D30, D40, D70, and D80, resistors R1 to R4, R7, and R8, and capacitors C1 to C4, C7, and C8.

The transistors Q1 to Q8 include Q1, Q3, Q5, and Q8 using P-channel Field Effect Transistors, and Q2, Q4, Q6, and Q7 using N-channel Field Effect Transistors. The transistors Q1 to Q6 form first Field Effect Transistors, and include complementary transistors in which transistor characteristics are the same in rating. The transistors Q7 and Q8 form second Field Effect Transistors, and, as will be described later, are operated only when electric charge charged to the piezoelectric transducer 11 is discharged. The second Field Effect Transistors require small current, and thus, are lower in the maximum rating of drain current as compared to the first Field Effect Transistors.

The transistors Q1 and Q2 form first complementary transistors, and configure a first push-pull circuit 41 without first rectification elements. In the first push-pull circuit 41, coupling of the positive and negative power source voltages ±HVH that are the maximum driving voltages coupled to source terminals of the transistors Q1 and Q2 to the output line 1 is controlled by on/off operation of the transistors Q1 and Q2. An electric signal for turning on or off the transistors Q1 and Q2 is formed by the first driver 34 of the pulsar control unit 32, and is input to gate terminals of the transistors Q1 and Q2 through the capacitors C1 and C2 through which AC coupling is performed. The gate terminals of the transistors Q1 and Q2 are coupled to the source terminals through the resistors R1 and R2 and protection diodes D1 and D2, respectively, and perform determination of the operation potential and overvoltage protection of the gate terminals. Drain terminals of the transistors Q1 and Q2 are coupled to each other, and serve as output units of the first push-pull circuit 41. The output units are coupled to the output line 1.

The transistors Q3 and Q4 form first complementary transistors, and configure a first push-pull circuit 42 with the first rectification elements. The first push-pull circuit 42 with the first rectification elements is a circuit to which voltage lower than the maximum driving voltage is supplied from the pulsar power source unit 31, and coupling of the positive and negative power source voltages ±HVL that are the intermediate driving voltages coupled to source terminals of the transistors Q3 and 4 to the output line 1 is controlled by on/off operation of the transistors Q3 and Q4. An electric signal for turning on or off the transistors Q3 and Q4 is formed by the first driver 34 of the pulsar control unit 32, and is input to gate terminals of the transistors Q3 and Q4 through the capacitors C3 and C4 through which AC coupling is performed. The gate terminals of the transistors Q3 and Q4 are coupled to the source terminals through the resistors R3 and R4 and protection diodes D3 and D4, respectively, and perform determination of the operation potential and overvoltage protection of the gate terminals.

The diodes D30 and D40 that are the first rectification elements couple between drain terminals of the transistors Q3 and Q4 and the output line 1, and the coupling portion therebetween serves as an output unit of the first push-pull circuit 42. When the voltage of the output line 1 is higher than the voltage +HVL of the source terminal of the transistor Q3, the diode D30 that is the first rectification element prevents reverse current flowing toward the side from which the voltage +HVL is supplied (toward the pulsar power source unit 31), from flowing into the transistor Q3. When the voltage of the output line 1 is lower than the voltage −HVL of the source terminal of the transistor Q4, the diode D40 that is the first rectification element prevents reverse current flowing toward the output line 1 side from flowing into the transistor Q4.

The transistors Q5 and Q6 form ground circuits that controls coupling of the ground terminals to the output line 1 by on/off operation of the transistors Q5 and Q6. A control signal that turns on or off the transistors Q5 and Q6 that are the ground circuits is formed by the pulsar control unit 32.

The transistors Q7 and Q8 form second complementary transistors, and configure a second push-pull circuit 43 with second rectification elements. In the second push-pull circuit 43, coupling of the positive and negative power source voltages ±HVL that are the intermediate driving voltages coupled to source terminals of the transistors Q7 and Q8 to the output line 1 is controlled by on/off operation of the transistors Q7 and Q8. In the example, the second push-pull circuit 43 couples the interconnected portion between the transistor Q3 in the first push-pull circuit 42 and the power source voltage +HVL to the output line 1, and couples the interconnected portion between the transistor Q4 in the first push-pull circuit 42 and the power source voltage −HVL to the output line 1. When reverse voltage is applied to the diodes D30 and D40, the second push-pull circuit 43 turns on the transistors Q7 and Q8 to allow current to flow in the direction opposite to that flowing the first push-pull circuit 42.

The transistors Q7 and Q8 are the N-channel and P-channel Field Effect Transistors, and require small current, for example, about half the maximum rating of the drain current, as compared to the transistors Q1 to Q4.

An electric signal for turning on or off the transistors Q7 and Q8 is formed by the second driver 35, with less driving capability, of the pulsar control unit 32, and is input to gate terminals of the transistors Q7 and Q8 through the capacitors C7 and C8 through which AC coupling is performed. The gate terminals of the transistors Q7 and Q8 are coupled to the source terminals through the resistors R7 and R8 and protection diodes D7 and D8, respectively, and perform determination of the operation potential and overvoltage protection of the gate terminals.

The diodes D70 and D80 that are the second rectification elements couple between drain terminals of the transistors Q7 and Q8 and the output line 1, and the coupling portion therebetween serves as an output unit of the second push-pull circuit 43. When the voltage of the output line 1 is higher than the voltage +HVL of the source terminal of the transistor Q7, the diode D70 that is the second rectification element is coupled to the transistor Q7 so as to flow current. When the voltage of the output line 1 is lower than the voltage −HVL of the source terminal of the transistor Q8, the diode D80 that is the second rectification element is coupled to the transistor Q8 so as to flow current.

The controlling signals from the pulsar control unit 32 to the transistors Q1 to Q8 of the multilevel pulsar 33 are represented by DVPH, DVNH, DVPL, DVPL*, DVNL, DVNL*, CPP, and CPN, respectively. In these character strings, DV is an abbreviation of Drive, N is an abbreviation of N-channel, P is an abbreviation of P-channel, H is an abbreviation of the maximum driving voltage HVH, and L is an abbreviation of the intermediate driving voltage HVL. In addition, the control signals, each having the mark * at the upper right of the character string, represent the control signals synchronized with DVPL and DVNL that are driven by the second driver 35.

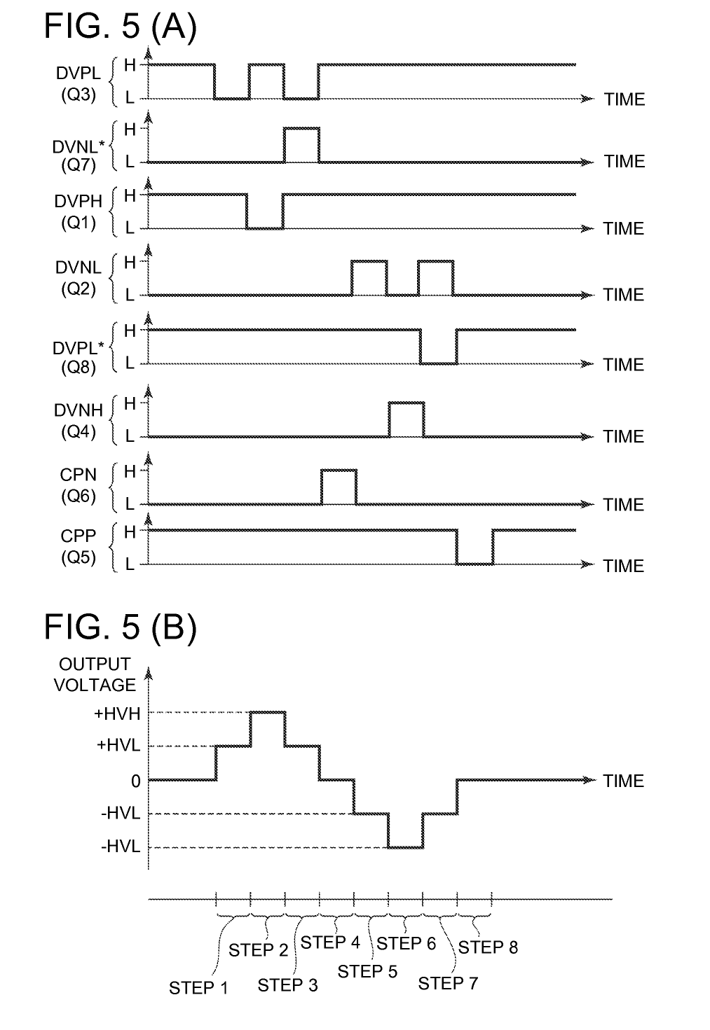

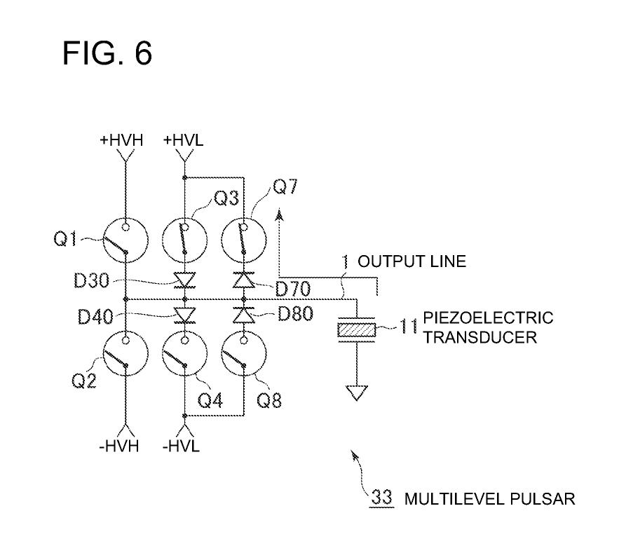

Next, the operation of the multilevel pulsar 33 will be described using FIGS. 5(A), 5(B), and 6. FIGS. 5(A) and 5(B) are diagrams for showing time changes of the control signals that drive the transistors Q1 to Q8 of the multilevel pulsar 33, and the pseudo sine waves to be output. The horizontal axis represents a time axis, and the vertical axis represents a voltage. It should be noted that the drawings shown in FIGS. 5(A) and 5(B) share the time axis.

DVPL, DVPH, CPP, and DVPL* that are the control signals of the transistors Q3, Q1, Q5, and Q8, respectively, using the P-channel Field Effect Transistors allow the transistors to be in on-states when the control signals are at L-levels of low voltage levels, and to be in off-states when the control signals are at H-levels of high voltage levels. Further, DVNL, DVNH, CPN, and DVNL* that are the control signals of the transistors Q2, Q4, Q6, and Q7, respectively, using the N-channel Field Effect Transistors allow the transistors to be in off-states when the control signals are at the L-levels of low voltage levels, and to be in off-states when the control signals are at the H-levels of high voltage levels.

In FIG. 5(A), DVPL of the control signal becomes the L-level, and the transistor Q3 becomes the on-state (Step 1). At this timing, the intermediate driving voltage +HVL is output as the output voltage of Step 1 shown in FIG. 5(B).

Thereafter, when DVPL of the control signal becomes the H-level, the transistor Q3 becomes the off-state, and at the same time, when DVPH of the control signal becomes the L-level, the transistor Q1 becomes the on-state (Step 2). At this timing, the maximum driving voltage +HVH is output as the output voltage of Step 2 shown in FIG. 5(B).

Thereafter, when DVPH of the control signal becomes the H-level, the transistor Q1 becomes the off-state, and at the same time, when DVPL of the control signal becomes the L-level, the transistor Q3 becomes the on-state (Step 3). At this timing, the intermediate driving voltage +HVL is output as the output voltage of Step 3 shown in FIG. 5(B), and at the same time, when DVNL* of the control signal becomes the H-level, the transistor Q7 becomes the off-state. It should be noted that the operation at this timing is described later in detail.

Thereafter, when DVPL and DVNL* of the control signals become the H-level and the L-level, respectively, the transistors Q3 and Q7 become the off-states, and at the same time, when CPN of the control signal becomes the H-level, the transistor Q6 becomes the on-state (Step 4). At this timing, the ground potential is output as the output voltage of Step 4 shown in FIG. 5(B).

Thereafter, when CPN of the control signal becomes the L-level, the transistor Q6 becomes the off-state, and at the same time, when DVNL of the control signal becomes the H-level, the transistor Q4 becomes the on-state (Step 5). At this timing, the negative intermediate driving voltage −HVL is output as the output voltage of Step 5 shown in FIG. 5(B).

Thereafter, when DVNL of the control signal becomes the L-level, the transistor Q4 becomes the off-state, and at the same time, when DVNH of the control signal becomes the H-level, the transistor Q2 becomes the on-state (Step 6). At this timing, the negative maximum driving voltage −HVH is output as the output voltage of Step 6 shown in FIG. 5(B).

Thereafter, when DVNH of the control signal becomes the L-level, the transistor Q2 becomes the off-state, and at the same time, when DVNL of the control signal becomes the H-level, the transistor Q4 becomes the on-state (Step 7). At this timing, the negative intermediate driving voltage −HVL is output as the output voltage of Step 7 shown in FIG. 5(B). In addition, at this timing, DVPL* of the control signal becomes the L-level, and the transistor Q8 becomes the on-state at the same time.

Thereafter, when DVNL and DVPL* of the control signals become the L-level and the H-level, respectively, the transistors Q4 and Q8 become the off-states, and at the same time, when CPP of the control signal becomes the L-level, the transistor Q5 becomes the on-state (Step 8). At this timing, the ground potential is output as the output voltage of Step 8 shown in FIG. 5(B).

With the above-described operation, one wavelength of the pseudo sine wave is formed. Thereafter, the operations of Steps 1 to 8 are repeated to form the burst waveform having the predetermined number of pseudo sine waves.

FIG. 6 is a diagram for schematically explaining a state of the circuit in which the transistor Q1 becomes the off-state, and the transistors Q3 and Q7 become the on-states in Step 3. In the drawing, the transistors Q1 to Q8 are illustrated as simplified on-off switches, and the illustration of the transistors Q5 and Q6 that are the ground circuits in the off-states is omitted.

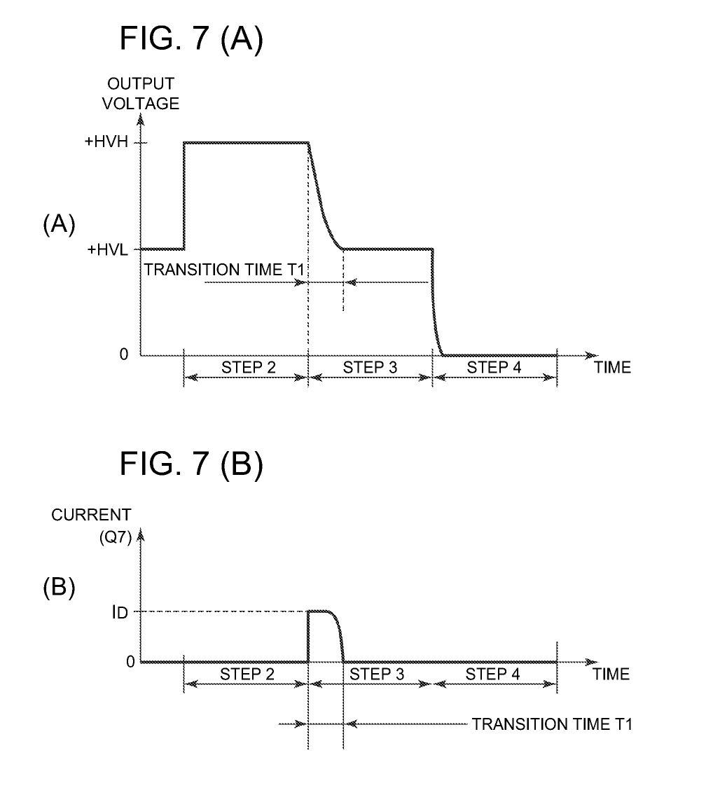

FIGS. 7(A) and 7(B) are diagrams for explaining enlarged voltage and current waveforms output to the output line 1 when Step 2 moves to Step 3. In FIG. 7(A), the horizontal axis represents time, and the vertical axis represents output voltage of the output line 1. In addition, in FIG. 7(B), the horizontal axis shares the time axis similar to FIG. 7(A), and the vertical axis represents the level of current flowing in the transistor Q7.

Here, in Step 2 that is the previous step of Step 3, the maximum driving voltage +HVH is output to the output line 1. In this state, electric charge corresponding to the applying voltage of +HVH is charged to the piezoelectric transducer 11 that is a capacitive load.

Thereafter, in Step 3, when the transistor Q1 becomes the off-state, the transistors Q3 and Q7 become the on-states as shown in FIG. 6. At this time, the voltage of +HVH is maintained in the output line 1 by the electric charge charged to the piezoelectric transducer 11, and the diode D30 becomes the off-state. In the meantime, the diode D70 becomes the on-state in which forward voltage is applied. In this state, the electric charge with the potential of +HVH charged to the piezoelectric transducer 11 is discharged, through the diode D7 and the transistor Q7, to the pulsar power source unit 31 which outputs the intermediate driving voltage +HVL.

The transistor Q7 is lower in the maximum rating of drain current as compared to the transistors Q1 to Q8, and thus, the current flowing in the transistor Q7 becomes substantially constant in the discharge. FIG. 7(B) is a diagram for showing the current flowing in the transistor Q7 when Step 2 moves to Step 3. During a transition time T1 when the electric potential of the electric charge charged to the piezoelectric transducer 11 is changed from +HVH to +HVL, substantially-constant drain current ID flows in the transistor Q7.

FIG. 7(A) shows a state in which the output voltage of the output line 1 is temporally changed. The output voltage is decreased from +HVH to +HVL in a substantially linear manner, and when the output voltage reaches +HVL, the diode D70 becomes the off-state in which reverse bias voltage is applied. At this time, the diode D30 becomes the on-state in which forward bias voltage is applied.

Even in the case where Step 6 moves to Step 7, the same operation is performed though the polarity of the voltage is inversed. In this case, when the voltage of the output line 1 is changed from the negative maximum driving voltage −HVH to the negative intermediate driving voltage −HVL, the diode D80 becomes the on-state. Accordingly, current flows from the transistor Q8 and the diode D80 to the piezoelectric transducer 11, and the electric charge charged to the piezoelectric transducer 11 is discharged only during the transition time.

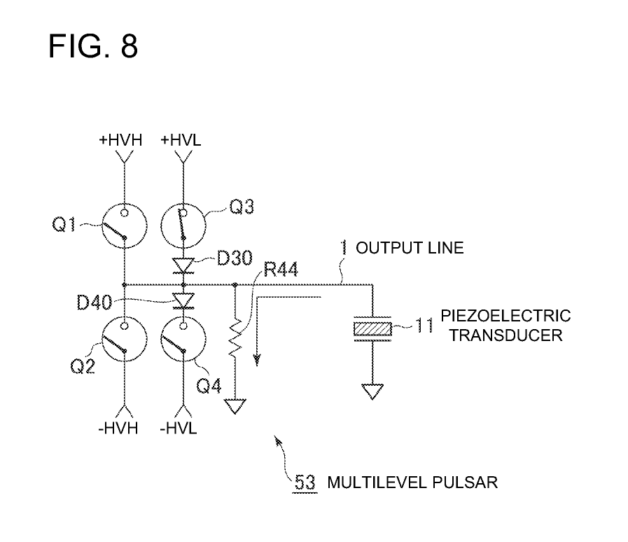

The electric power consumed by the multilevel pulsar 33 is smaller than that consumed by, for example, a multilevel pulsar 53 with a configuration shown below. FIG. 8 is a diagram for explaining a simplified configuration of the multilevel pulsar 53, as similar to FIG. 6. Transistors Q1 to Q4, diodes D30 and D40, power source voltages ±HVH and ±HVL, transistors Q5 and Q6 (not shown) that are ground circuits, and an output line 1 of the multilevel pulsar 53 are the same as those of the multilevel pulsar 33. In the multilevel pulsar 53, a resistor R44 that couples the output line 1 to a ground terminal is arranged in order to discharge electric charge charged to a piezoelectric transducer 11. Here, the value of the resistor R44 is about 100 to 300Ω.

Here, in Step 2 that is the previous step of Step 3, the maximum driving voltage +HVH is output to the output line 1, as similar to FIG. 6. In this state, electric charge corresponding to the applying voltage of +HVH is charged to the piezoelectric transducer 11 that is a capacitive load.

Thereafter, when the transistor Q1 becomes the off-state, the transistors Q3 becomes the on-state as shown in FIG. 8. At this time, the voltage of +HVH is maintained in the output line 1 by the electric charge charged to the piezoelectric transducer 11, and the diode D30 becomes the off-state. In this state, the electric charge charged to the piezoelectric transducer 11 passes through the resistor R44, so that current flows to the ground terminal, and transition current is generated during a transition time T2.

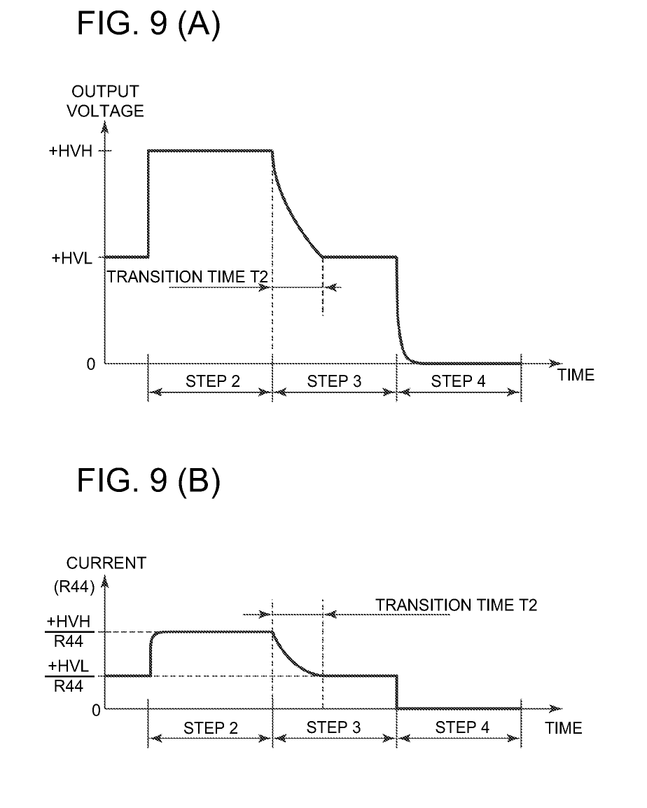

FIGS. 9(A) and 9(B) are diagrams for explaining an operation in the case of using the multilevel pulsar 53. In FIG. 9(A), the horizontal axis represents a time axis on which Step 2 moves to Step 3 and Step 4, and the vertical axis represents a voltage axis for showing changes in the output voltage of the multilevel pulsar 53. FIG. 9(B) shares the time axis similar to FIG. 9(A), and has the vertical axis showing current flowing in the resistor R44. The transition current flowing in the resistor R44 as shown in FIG. 8 flows during the transition time T2 when Step 2 moves to Step 3 in the voltage waveform of FIG. 9(A).

Thereafter, the output voltage of the output line 1 is decreased from +HVH to +HVL due to the discharge of the electric charge accumulated in the piezoelectric transducer 11. Here, during a period when the diode D30 becomes the on-state and the transistor Q3 becomes the on-state, the output line 1 is maintained at the intermediate driving voltage +HVL. In the voltage waveform shown in FIG. 9(A), the voltage of +HVL is output to the output line 1 during a period from the time when the transition time T2 passes in Step 3 to the time when Step 3 moves to Step 4. It should be noted that current +HVL/R44 flows in the resistor R44 during the period.

The power consumption of the multilevel pulsar 53 generated in Steps 1 to 8 is larger than that generated in the multilevel pulsar 33. Specifically, during a period when the output voltage in Steps 1 to 8 is not 0V, current that constantly flows in the resistor R44 is generated in the multilevel pulsar 53. The current increases the power consumption of the multilevel pulsar 53 using the resistor R44. In the meantime, current is not constantly consumed in the multilevel pulsar 33 except for a case where the piezoelectric transducer 11 is charged or discharged. In the case where the electric charge charged to the piezoelectric transducer 11 is discharged, it is possible to discharge at a high speed while turning on the transistor Q7 or Q8, so that the power consumption can be further decreased. The power consumption generated in the case of charging to the piezoelectric transducer 11 is the same in the multilevel pulsar 33 and the multilevel pulsar 53.

As described above, in the embodiment, there is provided the second push-pull circuit having the transistors Q7 and Q8 and the diodes D70 and D80 coupled between the intermediate driving voltage ±HVL and the output line 1, and the current that is constantly consumed in Steps 1 to 8 is eliminated to discharge the electric charge charged to the piezoelectric transducer 11 at a high speed. Accordingly, it is possible to reduce the power consumption and to reduce the heat generation of the multilevel pulsar 33.

The embodiment of the invention has been described above, and it is obvious that the invention can be variously changed and implemented in a range without changing the gist of the invention. For example, although not particularly shown, as a part of the ground circuit including the transistors Q5 and Q6 of the multilevel pulsar 33, a resistor that couples between the output line 1 and the ground terminal may be further provided. In this case, the value of the resistor is 500Ω or more that is larger as compared to the resistor R44 of the multilevel pulsar 53. Accordingly, it is possible to configure the multilevel pulsar in which increase of the power consumption is suppressed as compared to the multilevel pulsar 53 with a simple configuration.

In addition, the second Field Effect Transistors Q7 and Q8 are turned on or off while being synchronized with the first Field Effect Transistors Q3 and Q4 in the embodiment. However, the second Field Effect Transistors Q7 and Q8 may be turned on only for a predetermined period exceeding, for example, the transition time TI after the first Field Effect Transistor Q3 or Q4 is turned on.

In addition, the second Field Effect Transistors Q7 and Q8 are turned on or off while being synchronized with the first Field Effect Transistors Q3 and Q4 in the embodiment. However, in the case where the electric signal of the rectangular waveform is generated by turning on or off the first Field Effect Transistors Q1 and Q2 while maintaining the first Field Effect Transistors Q3 and Q4 in the off-states, it is possible not to operate the second Field Effect Transistors Q7 and Q8 without turning on them.

In addition, it is obvious that the circuit diagram showing the configuration of the multilevel pulsar 33 shown in FIG. 4 may be appropriately changed in a range without changing the gist of the invention.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus configured to supply a predetermined voltage to a piezoelectric transducer to transmit an ultrasonic wave, the ultrasonic imaging apparatus comprising:
a pulsar comprising an output line coupled to the piezoelectric transducer and a plurality of first push-pull circuits comprising output units that are coupled to the output line; and
a power source unit configured to supply a plurality of power source voltages with different levels to the plurality of first push-pull circuits, wherein at least one of the plurality of first push-pull circuits comprises first rectification elements configured to prevent reverse current from flowing into first complementary transistors configuring the first push-pull circuit, and wherein the pulsar further comprises a second push-pull circuit comprising an output unit that is coupled to the output, line such that when the same power source voltage as in the first push-pull circuit with the first rectification elements is applied to the second push-pull circuit, a current in a direction opposite to a current direction in the first push-pull circuit flows in the second push-pull circuit by turning on second complementary transistors configuring the second push-pull circuit.

2. The ultrasonic imaging apparatus according to claim 1, wherein the power source unit is configured to supply the maximum driving voltage among the plurality of power source voltages to the first push-pull circuit without the first rectification elements.

3. The ultrasonic imaging apparatus according to claim 1, wherein the plurality of first push-pull circuits comprises, as the first complementary transistors, P-channel first Field Effect Transistors on a high voltage side of the power source voltage relative to the output units, and N-channel Field Effect Transistors on a low voltage side of the power source voltage relative to the output units.

4. The ultrasonic imaging apparatus according to claim 2, wherein the plurality of first push-pull circuits comprises, as the first complementary transistors, P-channel first Field Effect Transistors on a high voltage side of the power source voltage relative to the output units, and N-channel Field Effect Transistors on a low voltage side of the power source voltage relative to the output units.

5. The ultrasonic imaging apparatus according to claim 3, wherein the second push-pull circuit comprises, as the second complementary transistors, an N-channel second Field Effect Transistor on the high voltage side relative to the output unit coupled to the output line, and a P-channel Field Effect Transistor on the low voltage side relative to the output unit, the second push-pull circuit further comprising second rectification elements which are coupled in series to the respective second Field Effect Transistors.

6. The ultrasonic imaging apparatus according to claim 4, wherein the second push-pull circuit comprises, as the second complementary transistors, an N-channel second Field Effect Transistor on the high voltage side relative to the output unit coupled to the output line, and a P-channel Field Effect Transistor on the low voltage side relative to the output unit, the second push-pull circuit further comprising second rectification elements which are coupled in series to the respective second Field Effect Transistors.

7. The ultrasonic imaging apparatus according to claim 5, wherein each of the second rectification elements which are coupled in series to the respective N-channel second Field Effect Transistors is a diode that is coupled in a forward direction from the output unit toward the power source unit, and each of the second rectification elements which are coupled in series to the respective P-channel second Field Effect Transistors is a diode that is coupled in the forward direction from the power source unit toward the output unit.

8. The ultrasonic imaging apparatus according to claim 6, wherein each of the second rectification elements which are coupled in series to the respective N-channel second Field Effect Transistors is a diode that is coupled in a forward direction from the output unit toward the power source unit, and each of the second rectification elements which are coupled in series to the respective P-channel second Field Effect Transistors is a diode that is coupled in the forward direction from the power source unit toward the output unit.

9. The ultrasonic imaging apparatus according to claim 5, further comprising a pulsar control unit configured to selectively activate and deactivate the first Field Effect Transistors and the second Field Effect Transistors.

10. The ultrasonic imaging apparatus according to claim 6, further comprising a pulsar control unit configured to selectively activate and deactivate the first Field Effect Transistors and the second Field Effect Transistors.

11. The ultrasonic imaging apparatus according to claim 7, further comprising a pulsar control unit configured to selectively activate and deactivate the first Field Effect Transistors and the second Field Effect Transistors.

12. The ultrasonic imaging apparatus according to claim 9, wherein the pulsar control unit comprises a first driver configured to selectively activate and deactivate the first Field Effect Transistors and a second driver configured to selectively activate and deactivate the second Field Effect Transistors.

13. The ultrasonic imaging apparatus according to claim 9, wherein the pulsar control unit comprises a pseudo sine wave generation device configured to output the plurality of power source voltages to the output line in a sine wave shape by selectively activating and deactivating the first Field Effect Transistors in a predetermined order.

14. The ultrasonic imaging apparatus according to claim 13, wherein the pseudo sine wave generation device is configured to activate one of the N-channel second Field Effect Transistor of the second push-pull circuit and the P-channel second Field Effect Transistor of the second push-pull circuit and to simultaneously deactivate one of the N-channel first Field Effect Transistor of the first push-pull circuit and the P-channel first Field Effect Transistor of the first push-pull circuit without the first rectification elements to activate one of the N-channel first Field Effect Transistor and the P-channel first Field Effect Transistor with the first rectification elements.

15. The ultrasonic imaging apparatus according to claim 14, wherein the pseudo sine wave generation device is configured to deactivate one of the N-channel second Field Effect Transistor and the P-channel second Field Effect Transistor and to simultaneously deactivate one of the N-channel first Field Effect Transistor and the P-channel first Field Effect Transistor with the first rectification elements.

16. The ultrasonic imaging apparatus according to claim 14, wherein the pseudo sine wave generation device is configured to deactivate one of the N-channel second Field Effect Transistor and the P-channel second Field Effect Transistor after a predetermined period of time passes after one of the N-channel first Field Effect Transistor and the P-channel first Field Effect Transistor without the first rectification elements is deactivated and one of the N-channel first Field Effect Transistor and the P-channel first Field Effect Transistor with the first rectification elements is activated.

17. The ultrasonic imaging apparatus according to claim 9, wherein the pulsar control unit is configured to selectively activate and deactivate only one of the N-channel first Field Effect Transistor and the P-channel first Field Effect Transistor without the first rectification elements to output the power source voltages to the output line in a rectangular shape without selectively activating and deactivating one of the N-channel second Field Effect Transistor and the P-channel second Field Effect Transistor.

18. The ultrasonic imaging apparatus according to claim 5, wherein the second Field Effect Transistors have a lower maximum rating of drain current flowing between drains and sources, as compared to the first Field Effect Transistors.

19. The ultrasonic imaging apparatus according to claim 1, wherein the power source unit is configured to generate the power source voltages with a same level and with positive and negative voltage polarities.

20. The ultrasonic imaging apparatus according to claim 1, wherein the pulsar further comprises a ground circuit which configured to selectively activate and deactivate the connection between the output line and a ground terminal.
